(19) United States
(12) Patent Application Publication
(10) Pub. No.: US 2024/0082333 A1
(43) Pub. Date: Mar. 14, 2024

(54) READILY DISSOLVABLE STEVIOL GLYCOSIDE COMPOSITIONS

(71) Applicant: CARGILL, INCORPORATED, Wayzata, MN (US)

(72) Inventors: Dan S. GASPARD, Victoria, MN (US); Adam T. ZARTH, St. Louis Park, MN (US)

(73) Assignee: CARGILL, INCORPORATED, Wayzata, MN (US)

(21) Appl. No.: 18/335,946

(22) Filed: Jun. 15, 2023

Related U.S. Application Data
(63) Continuation of application No. 16/753,869, filed on Apr. 6, 2020, now abandoned.
(60) Provisional application No. 62/569,279, filed on Oct. 6, 2017.
(60) Provisional application No. 62/676,722, filed on May 25, 2018.

(51) Int. Cl. A61K 36/28; A23F 3/34; A23L 2/56; A23L 2/60; A23L 2/68; A23L 5/40; A23L 27/00; A23L 27/30; A61K 31/192; A61K 31/235; B01D 15/36; C07H 15/256; C07K 1/16; C07K 1/18

(52) U.S. Cl. CPC A61K 36/28; A23F 3/34; A23L 2/56; A23L 2/60; A23L 2/68; A23L 5/40; A23L 27/30; A23L 27/36; A23L 27/39; A23L 27/88; A61K 31/192; A61K 31/235; B01D 15/361; C07H 15/256; C07K 1/16; C07K 1/18; A23V 2002/00; A23V 2200/15; A23V 2250/258; A61K 2236/31; A61K 2236/39

(57) ABSTRACT

A readily dissolvable steviol glycoside composition comprising a steviol glycoside and a dissolution enhancer compound in an amount effective to increase dissolution of the steviol glycoside.

### CROSS-REFERENCE TO RELATED APPLICATIONS

This application is a Continuation of U.S. application Ser. No. 16/753,869, filed Apr. 6, 2020, which is a national phase application of PCT Application No. PCT/US2018/054698, filed Oct. 5, 2018, which claims the benefit of U.S. Provisional Application No. 62/569,279, filed Oct. 6, 2017, and U.S. Provisional Application No. 62/676,722, filed May 25, 2018, each of which is incorporated by reference in its entirety.

### BACKGROUND

Steviol glycosides are glycosides of steviol, a diterpene compound and are about 150 to 450 times sweeter than sugar. Examples of steviol glycosides are described in WO 2013/096420 (see, e.g., listing in FIG. 1); and in Ohta et. al., “Characterization of Novel Steviol Glycosides from Leaves of Stevia rebaudiana Morita,” J. Appl. Glycosi., 57, 199-209 (2010) (See, e.g., Table 4 at p. 204). Structurally, the diterpene glycosides are characterized by a single base, steviol, and differ by the presence of carbohydrate residues at positions C13 and C19, as presented in FIGS. 2a-2k. See also PCT Patent Publication WO 20013/096420. Steviol glycosides can include one or more of dulcoside A, stevioside, and one or more of rebaudioside A, B, C, D, E, F, G, H, I, J, K, L, M, N, O, steviolbioside and rubusoside. Steviol glycosides have found use as non-caloric high intensity sweetener in foods and beverages.

### SUMMARY

The present disclosure generally relates to readily dissolvable steviol glycoside solutions comprising a steviol glycoside and a steviol glycoside dissolution enhancer compound (SG dissolution enhancer). One aspect provides a readily dissolvable steviol glycoside composition comprising a steviol glycoside and a dissolution enhancer compound in an amount effective to increase dissolution of the steviol glycoside, wherein the dissolution enhancer compound comprises at least one compound selected from the group consisting of a quinic acid, caffeic acid, ferulic acid, sinapic acid, p-coumaric acid, an ester of quinic acid, an ester of caffeic acid, an ester of ferulic acid, an ester of sinapic acid, an ester of p-coumaric acid, an ester of caffeic acid and quinic acid, an ester of caffeic acid and quinic acid comprising a single caffeic acid moiety, an ester of caffeic acid and quinic acid comprising more than one caffeic acid moiety, an ester of ferulic acid and quinic acid, an ester of ferulic acid and quinic acid comprising a single ferulic acid moiety, an ester of ferulic acid and quinic acid comprising more than one ferulic acid moiety, an ester of sinapic acid and quinic acid, an ester of sinapic acid and quinic acid comprising a single sinapic acid moiety, an ester of sinapic acid and quinic acid comprising more than one sinapic acid moiety, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety, a caffeic ester of 3-(3,4-dihydroxyphenyl)lactic acid, an ester of caffeic acid and tartaric acid, an ester of caffeic acid and tartaric acid comprising a single caffeic acid moiety, an ester of caffeic acid and tartaric acid comprising more than one caffeic acid moiety, salts thereof, and/or isomers thereof. In some aspects, the amount effective to increase dissolution of the steviol glycoside is an amount such that the dissolution enhancer compound comprises a 1:0.3 to 1:3 ratio by weight of steviol glycoside to dissolution enhancer compound. In other aspects, the amount effective to increase dissolution of the steviol glycoside is an amount such that the dissolution enhancer compound comprises a 1:1 to 1:3 ratio by weight of steviol glycoside to dissolution enhancer compound.

In some aspects, the dissolution enhancer compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, 3-O-feruloylquinic acid, 4-O-feruloylquinic acid, 5-O-feruloylquinic acid, 3,4-diferuloylquinic acid, 3,5-diferuloylquinic acid, 4,5-diferuloylquinic acid, rosmarinic acid, cichoric acid, caftaric acid, monocaffeoyltartaric acids, dicaffeoyltartaric acids and salts and/or isomers thereof. In other aspects, the dissolution enhancer compound comprises one or more compounds selected from the group consisting of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid. In some aspects, the dissolution enhancer compound comprises one or more compounds selected from the group consisting of 3-O-coumaroylquinic acid, 4-O-coumaroylquinic acid, 5-O-coumaroylquinic acid, 3,4-dicoumaroylquinic acid, 3,5-dicoumaroylquinic acid, and 4,5-dicoumaroylquinic acid.

In some aspects, the steviol glycoside is crystalline. In other aspects, the steviol glycoside comprises rebaudioside M. In some aspects, the steviol glycoside comprises rebaudioside D. In other aspects, the steviol glycoside comprises rebaudioside A.

In some aspects, the composition is a dry mixture. In other aspects, the composition is an admixture of steviol glycoside and dissolution enhancer compound. In some aspects, the composition is prepared by co-drying steviol glycoside and dissolution enhancer compound.

In some aspects, the dissolution enhancer compound is prepared from a botanical source. In other aspects, the botanical source is selected from the group consisting of eucommoia ulmoides, honeysuckle, Nicotiana benthamiana, globe artichoke, cardoon, stevia, Stevia rebaudiana, monkfruit, coffee, coffee beans, green coffee beans, tea, white tea, yellow tea, green tea, oolong tea, black tea, red tea, post-fermented tea, bamboo, heather, sunflower, blueberries, cranberries, bilberries, grouseberries, whortleberry, lingonberry, cowberry, huckleberry, grapes, chicory, eastern purple coneflower, echinacea, Eastern pellitory-of-the-wall, Upright pellitory, Lichwort, Greater celandine, Tetterwort, Nipplewort, Swallowwort, Bloodroot, Common nettle, Stinging nettle, Potato, Potato leaves, Eggplant, Aubergine, Tomato, Cherry tomato, Bitter apple, Thorn apple, Sweet potato, apple, Peach, Nectarine, Cherry, Sour cherry, Wild cherry, Apricot, Almond, Plum, Prune, Holly, Yerba mate, Mate, ilex paraguariensis, Guayusa, Yaupon Holly, Kuding, Guarana, Cocoa, Cocoa bean, Cacao, Cacao bean, Kola nut, Kola tree, Cola nut, Cola tree, Hornwort, Ostrich fern, Oriental ostrich fern, Fiddlehead fern, Shuttlecock fern, Oriental ostrich fern, Asian royal fern, Royal fern, Bracken, Brake, Common bracken, Eagle fern, Eastern brakenfern, dandelion, algae, seagrasses, Clove, Cinnamon, Indian bay leaf, Nutmeg, Bay laurel, Bay leaf, Basil, Great basil, Saint-Joseph's-wort, Thyme, Sage, Garden sage, Common sage, Culinary sage, Rosemary, Oregano, Wild marjoram, Marjoram, Sweet marjoram, Knotted marjoram, Pot marjoram, Dill, Anise, Star anise, Fennel, Florence fennel, Tarragon, Estragon, Mugwort, Licorice, Liquorice, Soy, Soybean, Soya bean, Wheat, Common wheat, Rice, Canola, Broccoli, Cauliflower, Cabbage, Bok choy, Kale, Collard greens, Brussels sprouts, Kohlrabi, Winter's bark, Elderflower, Assa-Peixe, Greater burdock, Valerian, and Chamomile. In some aspects, the botanical source is yerba mate. In other aspects, the botanical source is rosemary. In some aspects, the botanical source is chicory. In other aspects, the botanical source is stevia. In other aspects, the botanical source is globe artichoke. In some aspects, the botanical source is green coffee bean.

One aspect provides a readily dissolvable dry steviol glycoside composition comprising a steviol glycoside and a dissolution enhancer compound in an amount effective to increase dissolution of the steviol glycoside, wherein the dissolution enhancer compound comprises at least one caffeic ester of quinic acid, caffeic ester of 3-(3,4-dihydroxyphenyl)lactic acid, caffeic acid ester of tartaric acid, ferulic ester of quinic acid, and/or isomers thereof, and wherein the composition is a dry mixture. In some aspects, the dissolution enhancer compound comprises at least 15% dicaffeoylquinic acid. In other aspects, the amount effective to increase dissolution of the steviol glycoside is an amount such that the dissolution enhancer compound comprises a 1:0.3 to 1:3 ratio by weight of steviol glycoside to dissolution enhancer compound. In some aspects, the amount effective to increase dissolution of the steviol glycoside is an amount such that the dissolution enhancer comprises a 1:1 to 1:3 ratio by weight of steviol glycoside to dissolution enhancer. In other aspects, the composition comprises less than 0.3% (wt) of malonate, malonic acid, oxalate, oxalic acid, lactate, lactic acid, succinate, succinic acid, malate, or malic acid; or less than 0.05% (wt) of pyruvate, pyruvic acid, fumarate, fumaric acid, tartrate, tartaric acid, sorbate, sorbic acid, acetate, or acetic acid; or less than about 0.05% (wt) of chlorophyll.

In some aspects, the caffeic ester of quinic acid comprises at least one of chlorogenic acid, neochlorogenic acid, cryptochlorogenic acid, 3-O-caffeoylquinic acid, 4-O-caffeoylquinic acid, 5-O-caffeoylquinic acid, 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, or 4,5-dicaffeoylquinic acid. In other aspects, the ferulic ester of quinic acid comprises at least one of 3-O-feruloylquinic acid, 4-O-feruloylquinic acid, 5-O-feruloylquinic acid, 3,4-diferuloylquinic acid, 3,5-diferuloylquinic acid, or 4,5-diferuloylquinic acid. In some aspects, the caffeic ester of 3-(3,4-dihydroxyphenyl)lactic acid comprises rosmarinic acid. In other aspects, the caffeic acid ester of tartaric acid comprises cichoric acid. In other aspects, the caffeic acid ester of tartaric acid comprises caftaric acid.

In some aspects, the steviol glycoside is crystalline. In other aspects, the steviol glycoside comprises rebaudioside M. In some aspects, the steviol glycoside comprises rebaudioside D. In other aspects, the steviol glycoside comprises rebaudioside A. In some aspects, the dry composition is an admixture of steviol glycoside and dissolution enhancer compound. In other aspects, the dry composition is prepared by co-drying steviol glycoside and dissolution enhancer compound.

In some aspects, the dissolution enhancer compound is prepared from a botanical source. In other aspects, the botanical source is selected from the group consisting of eucommoia ulmoides, honeysuckle, Nicotiana benthamiana, globe artichoke, cardoon, stevia, Stevia rebaudiana, monkfruit, coffee, coffee beans, green coffee beans, tea, white tea, yellow tea, green tea, oolong tea, black tea, red tea, post-fermented tea, bamboo, heather, sunflower, blueberries, cranberries, bilberries, grouseberries, whortleberry, lingonberry, cowberry, huckleberry, grapes, chicory, eastern purple coneflower, echinacea, Eastern pellitory-of-the-wall, Upright pellitory, Lichwort, Greater celandine, Tetterwort, Nipplewort, Swallowwort, Bloodroot, Common nettle, Stinging nettle, Potato, Potato leaves, Eggplant, Aubergine, Tomato, Cherry tomato, Bitter apple, Thorn apple, Sweet potato, apple, Peach, Nectarine, Cherry, Sour cherry, Wild cherry, Apricot, Almond, Plum, Prune, Holly, Yerba mate, Mate, ilex paraguariensis, Guayusa, Yaupon Holly, Kuding, Guarana, Cocoa, Cocoa bean, Cacao, Cacao bean, Kola nut, Kola tree, Cola nut, Cola tree, Hornwort, Ostrich fern, Oriental ostrich fern, Fiddlehead fern, Shuttlecock fern, Oriental ostrich fern, Asian royal fern, Royal fern, Bracken, Brake, Common bracken, Eagle fern, Eastern brakenfern, dandelion, algae, seagrasses, Clove, Cinnamon, Indian bay leaf, Nutmeg, Bay laurel, Bay leaf, Basil, Great basil, Saint-Joseph's-wort, Thyme, Sage, Garden sage, Common sage, Culinary sage, Rosemary, Oregano, Wild marjoram, Marjoram, Sweet marjoram, Knotted marjoram, Pot marjoram, Dill, Anise, Star anise, Fennel, Florence fennel, Tarragon, Estragon, Mugwort, Licorice, Liquorice, Soy, Soybean, Soya bean, Wheat, Common wheat, Rice, Canola, Broccoli, Cauliflower, Cabbage, Bok choy, Kale, Collard greens, Brussels sprouts, Kohlrabi, Winter's bark, Elderflower, Assa-Peixe, Greater burdock, Valerian, and Chamomile. In some aspects, the botanical source is yerba mate, rosemary, chicory, globe artichoke, green coffee bean, and/or stevia.

### DETAILED DESCRIPTION

This disclosure relates generally to readily dissolvable steviol glycoside compositions comprising a steviol glycoside and a steviol glycoside dissolution enhancer compound (SG dissolution enhancer) in an amount effective to increase dissolution of the steviol glycoside.

An example of a readily dissolvable steviol glycoside composition is a composition comprising a steviol glycoside and a dissolution enhancer compound in an amount effective to increase dissolution of the steviol glycoside.

In some aspects, the term dissolution refers to the process of dissolving a solute (e.g., a steviol glycoside composition) into a solvent (e.g., a primarily aqueous solution) to make a solution (e.g., a steviol glycoside solution). Dissolution can also be thought of in terms of rate at which a solute dissolves into a solvent to make a solution. For example, the rate of dissolution or dissolution rate refers to a rate at which a solute dissolves into a solvent to make a solution (e.g., the rate at which a steviol glycoside composition dissolves into a primarily aqueous solution to make a steviol glycoside solution). The rate of solution can also be used to refer to a rate at which a solute interacts with a solvent to form a solution. In some aspects, rate of dissolution and rate of solution can be used interchangeably. In other aspects, the term instantaneous solubility can refer to having a high rate of dissolution and/or a high rate of solution. For example, instantaneous solubility can refer to having a high initial dissolution of a steviol glycoside compound into a primarily aqueous solution upon mixing.

In some aspects, the term readily dissolvable composition refers to a composition with a high rate of dissolution into certain solvents. A readily dissolvable composition can exhibit a high rate of dissolution or a high rate of solution into certain solvents. A readily dissolvable composition can also comprise instantaneous solubility into certain solvents. For example, a readily dissolvable steviol glycoside composition can comprise a high rate of dissolution into a primarily aqueous solution to yield a steviol glycoside solution. A readily dissolvable steviol glycoside composition can also have a high instantaneous solubility into a primarily aqueous solution to yield a steviol glycoside solution.

The steviol glycoside composition can include one or more steviol glycosides. Exemplary steviol glycosides include rebaudioside M, rebaudioside D, rebaudioside A, rebaudioside B, reaudioside O, and rebaudioside N. In some aspects, the one or more of the steviol glycosides are produced by fermentation by an engineered organism. For example, rebaudioside D and M can be produced by an engineered organism and then isolated to produce a steviol glycoside composition of primarily rebaudioside D and rebaudioside M as the predominant steviol glycoside species. In other aspects, the steviol glycoside composition can comprise rebaudioside D and rebaudioside M in an amount greater than other steviol glycosides. In some aspects, one or more of the steviol glycosides are isolated from Stevia rebaudiana.

The steviol glycoside composition can include one or more steviol glycosides. In some aspects, the term steviol glycoside refers to Rebaudioside A (Reb A) (CAS #58543-16-1), Rebaudioside B (Reb B) (CAS #58543-17-2), Rebaudioside C (Reb C) (CAS #63550-99-2), Rebaudioside D (Reb D) (CAS #63279-13-0), Rebaudioside E (Reb E) (CAS #63279-14-1), Rebaudioside F (Reb F) (CAS #438045-89-7), Rebaudioside M (Reb M) (CAS #1220616-44-3), Rubusoside (CAS #63849-39-4), Dulcoside A (CAS #64432-06-0), Rebaudioside I (Reb I) (MassBank Record: FU000332), Rebaudioside Q (Reb Q), Rebaudioside O (Reb O), Rebaudioside N (Reb N) (CAS #1220616-46-5), 1,2-Stevioside (CAS #57817-89-7), 1,3-Stevioside (Reb G), Steviol-1,2-Bioside (MassBank Record: FU000299), Steviol-1,3-Bioside, Steviol-13-O-glucoside (13-SMG), Steviol-19-O-glucoside (19-SMG), and steviol glycoside having 1, 2, 3, 4, 5, 6, 7, 8, 9, 10 or sugar additions (e.g., glucose, rhamnose, and/or xylose), and isomers thereof. See FIG. 1; see also, Steviol Glycosides Chemical and Technical Assessment 69th JECFA, 2007, prepared by Harriet Wallin, Food Agric. Org.

In some aspects, the steviol glycoside composition can optionally be described in terms of amounts of rebaudioside M and rebaudioside D. For example, rebaudioside M and rebaudioside D can be present in the composition in a total amount of about 80% (wt) or greater, 90% (wt) or greater, 95% (wt), 99% (wt) or greater, of a total amount steviol glycosides in the composition. Rebaudioside M can be the predominant steviol glycoside in the composition, and can be present, for example, in an amount in the range of about 50% to about 95%, about 70% to about 90%, or about 75% to about 85% of the total amount steviol glycosides in the composition. Rebaudioside D can be in an amount less than Rebaudioside M, such as in an amount in the range of about 5% to about 25%, about 10% to about 20%, or about 10% to about 15% of the total amount steviol glycosides in the composition. In other aspects, a steviol glycoside composition comprising about 80% (wt) rebaudioside M can be referred to as RM80. The composition can also optionally be expressed in terms of amounts of other known steviol glycosides that are present in lower amounts. For example, the composition can comprise mostly rebaudioside M and/or D and can include one or more of rebaudioside A, rebaudioside B, or stevioside in an amount of about 5% (wt) or less, about 2% (wt) or less, or about 1% (wt) or less, of a total amount steviol glycosides in the composition.

In some aspects, the steviol glycoside composition can optionally be described in terms of amounts of rebaudioside A. For example, rebaudioside A can be present in the composition in a total amount of about 80% (wt) or greater, 85% (wt) or greater, 90% (wt) or greater, 95% (wt) or greater, 98% (wt) or greater of a total amount of steviol glycosides in the composition. Rebaudioside A can be the predominant steviol glycoside in the composition, and can be present, for example, in an amount in the range of about 50% to about 98%, about 70% to about 98%, or about 90% to about 98% of the total amount steviol glycosides in the composition. Other rebaudiosides can be present in an amount less than Rebaudioside A, such as in an amount in the range of about 1% to about 40%, about 1% to about 20%, or about 10% to about 15% of the total amount steviol glycosides in the composition. In other aspects, a steviol glycoside composition comprising about 95% (wt) rebaudioside A can be referred to as RA95.

In some aspects, the steviol glycoside is in a crystalline form. The term crystalline form can refer to steviol glycoside that comprises individual macroscopic crystals of steviol glycoside wherein the individual macroscopic crystals comprise steviol glycoside structured into a crystal lattice. The term crystalline form can also refer to steviol glycoside that comprises polycrystals of steviol glycoside wherein the polycrystals comprise many crystals of steviol glycoside. In some aspects, steviol glycoside in crystalline form can be produced by an organic solvent crystallization of a steviol glycoside solution. In other aspects, steviol glycoside in crystalline form can be produced by an aqueous crystallization of a steviol glycoside solution. In other aspects, purification of steviol glycoside from crude steviol glycoside extracts results in a purified steviol glycoside in a crystalline form. A crystalline form comprising crystals with an ordered lattice structure is contrasted with an amorphous form in which the solid has no periodic arrangement of the molecules.

In some aspects, steviol glycoside in a crystalline form can have a reduced rate of dissolution in a primarily aqueous solution when compared to an amorphous form. Steviol glycoside in a crystalline form can have a reduced rate of solution in a primarily aqueous solution. Steviol glycoside in a crystalline form can also possess low instantaneous solubility in a primarily aqueous solution.

Examples of steviol glycoside stabilizing compounds include: caffeic acid, an ester of caffeic acid, an ester of caffeic acid and quinic acid, an ester of caffeic acid and quinic acid comprising a single caffeic acid moiety (e.g., chlorogenic acid, cryptochlorogenic acid, and neochlorogenic acid; structures of each are provided herein), an ester of caffeic acid and quinic acid comprising more than one caffeic acid moiety (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid; structures of each are provided herein); ferulic acid, an ester of ferulic acid, an ester of ferulic acid and quinic acid, an ester of ferulic acid and quinic acid comprising a single ferulic acid moiety, an ester of ferulic acid and quinic acid comprising more than one ferulic acid moiety; quinic acid, an ester of quinic acid; tartaric acid, a tartaric acid derivative, an ester of tartaric acid (e.g. caftaric acid or cichoric acid), an ester of a tartaric acid derivative, 3-(3,4-dihydroxyphenyl)lactic acid, a 3-(3,4-dihydroxyphenyl)lactic acid derivative, an ester of 3-(3,4-dihydroxyphenyl)lactic acid (e.g. rosmarinic acid), an ester of a 3-(3,4-dihydroxyphenyl)lactic acid derivative, p-coumaric acid, an ester of p-coumaric acid, an ester of p-coumaric acid and quinic acid, an ester of p-coumaric acid and quinic acid comprising a single p-coumaric acid moiety, an ester of p-coumaric acid and quinic acid comprising more than one p-coumaric acid moiety; sinapic acid, an ester of sinapic acid, an ester of sinapic acid and quinic acid, an ester of sinapic acid and quinic acid comprising a single sinapic acid moiety, an ester of sinapic acid and quinic acid comprising more than one sinapic acid moiety; and 3-O-feruloylquinic acid, 4-O-feruloylquinic acid, 5-O-feruloylquinic acid, 3,4-diferuloylquinic acid, 3,5-diferuloylquinic acid, and 4,5-diferuloylquinic acid.

Caffeic acid has the structure:

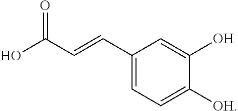 [SMILES: O=C(O)/C=C/c1ccc(O)c(O)c1]

Ferulic acid has the structure:

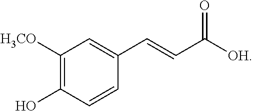 [SMILES: COc1cc(/C=C/C(=O)O)ccc1O]

p-Coumaric acid has the structure:

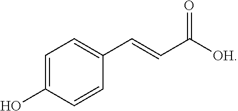 [SMILES: O=C(O)/C=C/c1ccc(O)cc1]

Sinapic acid has the structure:

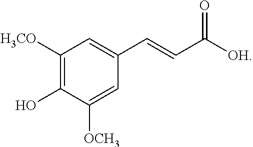 [SMILES: COc1cc(/C=C/C(=O)O)cc(OC)c1O]

Quinic acid has the structure:

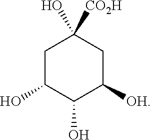 [SMILES: O=C(O)[C@]1(O)C[C@@H](O)[C@@H](O)[C@H](O)C1]

3-(3,4-dihydroxyphenyl)lactic acid has the structure:

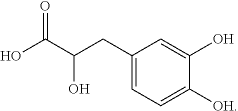 [SMILES: O=C(O)C(O)Cc1ccc(O)c(O)c1]

Tartaric acid has the structure:

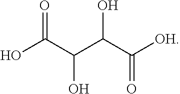

and can be in the D and L forms.

Examples of the esters of the various acids contemplated herein include the ester of caffeic acid and quinic acid, which includes monocaffeoylquinic acids (e.g., chlorogenic acid, neochlorogenic acid, and cryptochlorogenic acid), and dicaffeoylquinic acids (e.g., 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, and 4,5-dicaffeoylquinic acid), and salts thereof:

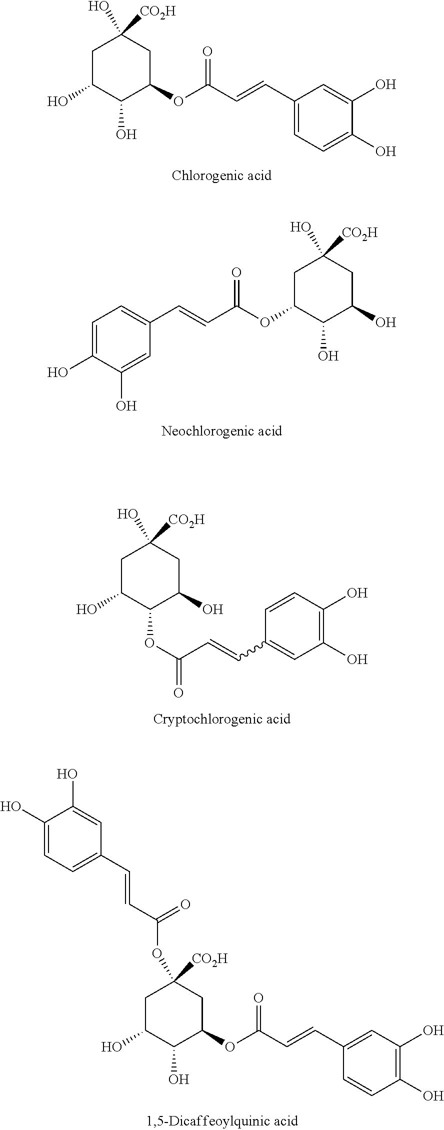 [SMILES: O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@@H]1C[C@@](O)(C(=O)O)C[C@@H](O)[C@@H]1O ; O=C(C=Cc1ccc(O)c(O)c1)O[C@H]1[C@H](O)C[C@](O)(C(=O)O)C[C@H]1O ; O=C(/C=C/c1ccc(O)c(O)c1)O[C@@H]1C[C@](OC(=O)/C=C/c2ccc(O)c(O)c2)(C(=O)O)C[C@@H](O)[C@H]1O]

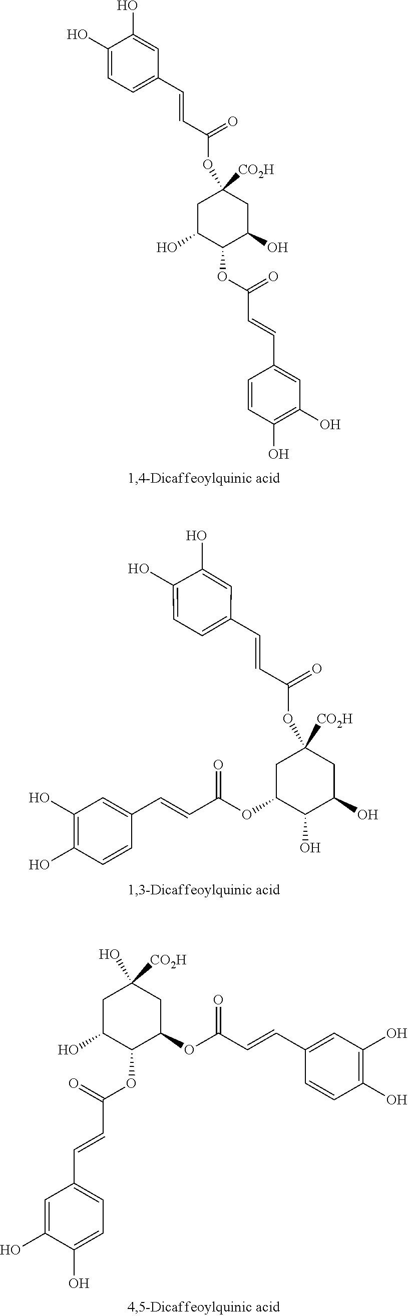 [SMILES: O=C(/C=C/c1ccc(O)c(O)c1)O[C@H]1[C@H](O)C[C@](OC(=O)/C=C/c2ccc(O)c(O)c2)(C(=O)O)C[C@H]1O ; O=C(/C=C/c1ccc(O)c(O)c1)O[C@@H]1C[C@@](OC(=O)/C=C/c2ccc(O)c(O)c2)(C(=O)O)C[C@@H](O)[C@@H]1O ; O=C(/C=C/c1ccc(O)c(O)c1)O[C@@H]1[C@H](O)C[C@@](O)(C(=O)O)C[C@H]1OC(=O)/C=C/c1ccc(O)c(O)c1]

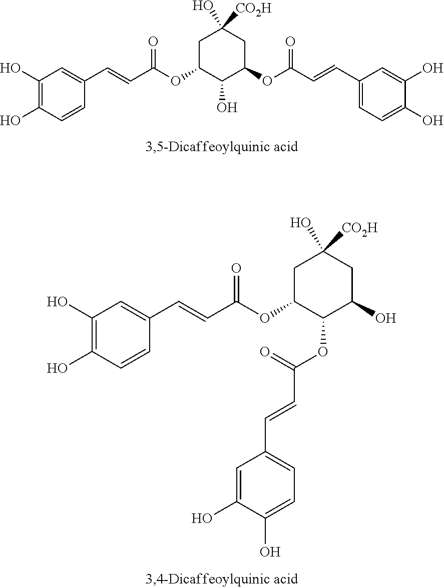 [SMILES: O=C(/C=C/c1ccc(O)c(O)c1)O[C@@H]1C[C@](O)(C(=O)O)C[C@@H](OC(=O)/C=C/c2ccc(O)c(O)c2)[C@H]1O ; O=C(/C=C/c1ccc(O)c(O)c1)O[C@H]1[C@H](O)C[C@](O)(C(=O)O)C[C@H]1OC(=O)/C=C/c1ccc(O)c(O)c1]

with 4,5-dicaffeoylquinic acid, 3,5-dicaffeoylquinic acid, and 3,4-dicaffeoylquinic acid being most prevalent in the compositions contemplated herein and most prevalent in abundant in stevia, yerba mate, globe artichoke, and green coffee bean.

Examples of the esters of the various acids contemplated herein include the ester of caffeic acid and tartaric acid, which includes cichoric acid having the structure:

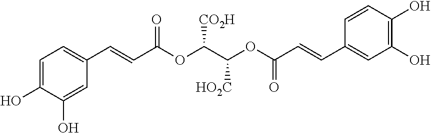 [SMILES: O=C(/C=C/c1ccc(O)c(O)c1)O[C@H](C(=O)O)[C@H](OC(=O)/C=C/c1ccc(O)c(O)c1)C(=O)O]

which has two caffeic acid molecules linked to a tartaric acid core; and caftaric acid having the structure:

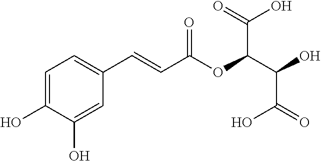 [SMILES: O=C(/C=C/c1ccc(O)c(O)c1)O[C@@H](C(=O)O)[C@@H](O)C(=O)O]

which has one caffeic acid molecule linked to a tartaric acid core.

Examples of the esters of the various acids contemplated herein include the ester of caffeic acid and 3-(3,4-dihydroxyphenyl)lactic acid including, for example, rosmarinic acid, which has the structure:

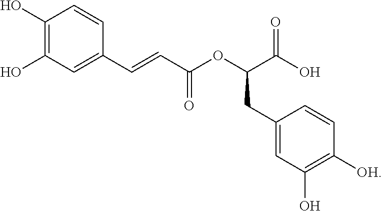 [SMILES: O=C(/C=C/c1ccc(O)c(O)c1)O[C@H](Cc1ccc(O)c(O)c1)C(=O)O]

Each of the caffeic acid, monocaffeoylquinic acids, dicaffeoylquinic acids and other dissolution enhancer compounds can be considered weak acids and can each exist in at least one of their conjugate acid form, conjugate base form (e.g., in their salt form), and mixed conjugate acid-conjugate base form, wherein a fraction (e.g., mole fraction) of the compounds exist in the conjugate acid form and another fraction exist in the conjugate base form. The fraction of conjugate acid form to conjugate base form for the caffeic acid, monocaffeoylquinic acids, dicaffeoylquinic acids, and other dissolution enhancer compounds will depend on various factors, including the pKa of each compound and the pH of the composition.

Examples of salts of caffeic acid, monocaffeoylquinic acids, dicaffeoylquinic acids, and other dissolution enhancer compounds include, but are not limited to, quaternary ammonium, sodium, potassium, lithium, magnesium, and calcium salts of caffeic acid, monocaffeoylquinic acids, dicaffeoylquinic acids, monoferuloylquinic acids, and diferuloylquinic acids, and other dissolution enhancer compounds and the like.

In some aspects, the dissolution enhancer compound can be enriched for one or more of caffeic acid, monocaffeoylquinic acids, and dicaffeoylquinic acids. The term “enriched” refers to an increase in an amount of one of caffeic acid, monocaffeoylquinic acids, and dicaffeoylquinic acids relative to one or more other compounds that are present in the dissolution enhancer compound. A dissolution enhancer compound that is enriched for one or more of caffeic acid, monocaffeoylquinic acids, and dicaffeoylquinic acids can increase dissolution of the steviol glycoside composition.

In some aspects, a dissolution enhancer compound enriched for one or more dicaffeoylquinic acids can increase dissolution of the readily dissolvable steviol glycoside composition. A dissolution enhancer compound that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more dicaffeoylquinic acids. In other aspects, a dissolution enhancer compound that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more of a combination of one or more of 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid, and salts thereof.

In some aspects, the dissolution enhancer compound comprises one or more compounds selected from the group consisting of 3-O-coumaroylquinic acid, 4-O-coumaroylquinic acid, 5-O-coumaroylquinic acid, 3,4-dicoumaroylquinic acid, 3,5-dicoumaroylquinic acid, 4,5-dicoumaroylquinic acid.

In some aspects, the dissolution enhancer compound may be isolated from botanical sources. Various botanical sources comprise dissolution enhancer compound and may be used to isolate dissolution enhancer compounds. Some examples of botanical sources from which dissolution enhancer compound may be isolated include yerba mate plant (Ilex paraguariensis), stevia, coffee, tea, chicory, and globe artichoke. Some botanical sources may produce dissolution enhancer compound that is enriched for one or more of caffeic acid, monocaffeoylquinic acids, and dicaffeoylquinic acids and can increase dissolution of steviol glycoside composition. For example, dissolution enhancer compound isolated from yerba mate plant is enriched for dicaffeoylquinic acids and can increase dissolution of the readily dissolvable steviol glycoside composition. In other aspects, dissolution enhancer compound isolated from yerba mate plant that is enriched for dicaffeoylquinic acids can comprise 10% or more, 15% or more, 20% or more, 25% or more, 30% or more, 35% or more, 40% or more, 45% or more, or 50% or more, 60% or more, 70% or more, or 80% or more, or 90% or more of a combination of one or more of 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid, and salts thereof.

In some aspects, an amount of dissolution enhancer compound effective to increase dissolution of the steviol glycoside is an amount such that the dissolution enhancer compound comprises a 1:0.3 to 1:3 ratio by weight of steviol glycoside to dissolution enhancer compound. In other aspects, an amount of dissolution enhancer compound effective to increase dissolution of the steviol glycoside is an amount such that the dissolution enhancer compound comprises a 1:1 to 1:3 ratio by weight of steviol glycoside to dissolution enhancer compound. An amount of dissolution enhancer compound effective to increase dissolution of the steviol glycoside can be an amount such that the dissolution enhancer compound comprises a ratio by weight of steviol glycoside to dissolution enhancer compound of 1:0.1 to 1:10. In some aspects an amount of dissolution enhancer compound effective to increase dissolution of the steviol glycoside can be an amount such that the dissolution enhancer compound comprises a ratio by weight of steviol glycoside to dissolution enhancer compound of about 1:0.1 to 1:5, about 1:0.5 to 1:4, about 1:0.3 to 1:3, or about 1:1 to 1:3. In other aspects an amount of dissolution enhancer compound effective to increase dissolution of the steviol glycoside can be an amount such that the dissolution enhancer compound comprises a ratio by weight of steviol glycoside to dissolution enhancer compound of about 1:0.1, 1:0.5, 1:1, 1:2, 1:3, 1:4, 1:5, 1:6, 1:7, 1:8, 1:9, or 1:10 by weight. In some aspects, an amount of dissolution enhancer compound effective to increase dissolution of the steviol glycoside can be an amount such that the dissolution enhancer compound comprises a ratio by weight of steviol glycoside to dissolution enhancer compound of about 1:0.3 to 1:3.

In some aspects, the readily dissolvable steviol glycoside composition comprises an increased rate of dissolution in a primarily aqueous solution when compared to a steviol glycoside composition without a dissolution enhancer compound. The dissolution rate can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the dissolution rate can be increased by 1, 2, 3, 4, 5, 6, 7, 8, 9, or 10 times or more when compared to a composition without a dissolution enhancer compound.

In some aspects, the readily dissolvable steviol glycoside composition comprises an increased rate of solution in a primarily aqueous solution when compared to a steviol glycoside composition without a dissolution enhancer compound. The rate of solution can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the rate of solution can be increased by 1, 2, 3, 4, 5, 6, 7, 8, 9, or 10 times or more when compared to a composition without a dissolution enhancer compound.

In some aspects, the readily dissolvable steviol glycoside composition comprises an instantaneous solubility in a primarily aqueous solution when compared to a steviol glycoside composition without a dissolution enhancer compound. The instantaneous solubility can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a steviol glycoside composition without a dissolution enhancer compound. In other aspects, the instantaneous solubility can be increased by 1, 2, 3, 4, 5, 6, 7, 8, 9, or 10 times or more when compared to a steviol glycoside composition without a dissolution enhancer compound.

In some aspects, the readily dissolvable steviol glycoside composition comprises an increased dissolution when dissolved in a primarily aqueous solution comprises primarily water. The primarily aqueous solution can also comprise less than 1%, 5%, 10%, 15%, 20%, 30%, 40%, 45%, 50%, 55%, 60%, 65%, 70%, 80%, or 90%, or about 40% to 65%, or about 50% to 55%, or about 55% of C1-C4 alcohol (e.g., methanol, ethanol, propanol, butanol, etc.). The primarily aqueous solution can be substantially free of C1-C4 alcohols. In other aspects, the primarily aqueous solution is essentially free of C1-C4 alcohols. In some aspects, the primarily aqueous solution comprises less than 1% stevioside. The primarily aqueous solution can comprise less than 3% rebaudioside B. The primarily aqueous solution can comprise less than 1% steviolbioside. The primarily aqueous solution can comprise less than 1% 13-SMG. In other aspects, the primarily aqueous solution comprises less than one or more of 1% stevioside, 1% rebaudioside B, 1% steviolbioside, and 1% 13-SMG. In some aspects, the primarily aqueous solution has any suitable pH. The primarily aqueous solution can also comprise a pH of 0, 1, 2, 3, 4, 5, or 6. The primarily aqueous solution can comprise a pH of between 0 and 7. The primarily aqueous solution can comprise a pH of between 1 and 6. The primarily aqueous solution can comprise a pH of between 1.5 and 4.

In some aspects, the readily dissolvable steviol glycoside composition comprises an increased rate of solution in a primarily aqueous solution when compared to a steviol glycoside composition without a dissolution enhancer compound when a final concentration of steviol glycoside is between 3000 ppm and 60000 ppm. The final concentration of steviol glycoside can be greater than 100 ppm, 200 ppm, 300 ppm, 400 ppm, 500 ppm, 600 ppm, 700 ppm, 800 ppm, 900 ppm, or 1000 ppm. The final concentration of steviol glycoside can be greater than 1100 ppm, 1200 ppm, 1300 ppm, 1400 ppm, 1500 ppm, 1600 ppm, 1700 ppm, 1800 ppm, or 1900 ppm. The final concentration of steviol glycoside can be greater than 2100 ppm, 2200 ppm, 2300 ppm, 2400 ppm, 2500 ppm, 2600 ppm, 2700 ppm, 2800 ppm, or 2900 ppm. The final concentration of steviol glycoside can be greater than 3100 ppm, 3200 ppm, 3300 ppm, 3400 ppm, 3500 ppm, 3600 ppm, 3700 ppm, 3800 ppm, or 3900 ppm. The final concentration of steviol glycoside can be greater than 3000 ppm, 4000 ppm, 5000 ppm, 6000 ppm, 7000 ppm, 8000 ppm, or 9000 ppm. The final concentration of steviol glycoside can be greater than 1%, 2%, 3%, 4%, 5%, 6%, 7%, 8%, 9%, 10%, or higher.

In some aspects, the readily dissolvable steviol glycoside composition comprises an increased rate of solution in a primarily aqueous solution when compared to a steviol glycoside composition without a dissolution enhancer compound when a final concentration of dissolution enhancer compound is between 3000 ppm and 60000 ppm. The final concentration of dissolution enhancer compound can be greater than 100 ppm, 200 ppm, 300 ppm, 400 ppm, 500 ppm, 600 ppm, 700 ppm, 800 ppm, 900 ppm, or 1000 ppm. The final concentration of dissolution enhancer compound can be greater than 1100 ppm, 1200 ppm, 1300 ppm, 1400 ppm, 1500 ppm, 1600 ppm, 1700 ppm, 1800 ppm, or 1900 ppm. The final concentration of dissolution enhancer compound can be greater than 2100 ppm, 2200 ppm, 2300 ppm, 2400 ppm, 2500 ppm, 2600 ppm, 2700 ppm, 2800 ppm, or 2900 ppm. The final concentration of dissolution enhancer compound can be greater than 3100 ppm, 3200 ppm, 3300 ppm, 3400 ppm, 3500 ppm, 3600 ppm, 3700 ppm, 3800 ppm, or 3900 ppm. The final concentration of dissolution enhancer compound can be greater than 3000 ppm, 4000 ppm, 5000 ppm, 6000 ppm, 7000 ppm, 8000 ppm, or 9000 ppm. The final concentration of dissolution enhancer compound can be greater than 1%, 2%, 3%, 4%, 5%, 6%, 7%, 8%, 9%, 10%, or higher.

In some aspects, the readily dissolvable steviol glycoside composition comprises an increased rate of solution in a primarily aqueous solution when compared to a steviol glycoside composition without a dissolution enhancer compound when the dissolution is carried out at a temperature less than 5° C., 10° C., 15° C., 20° C., 25° C., 30° C., 35° C., 40° C., 45° C., 50° C., 55° C., 60° C., 65° C., 70° C., 75° C., 80° C., or 85° C. The dissolution can be carried out at between 5° C. and 65° C. The dissolution can be carried out between 20° C. and 65° C. The dissolution can be carried out at between 10° C. and 40° C. The dissolution can be carried out at between 15° C. and 30° C. The dissolution can be carried out at between 20° C. and 25° C. The dissolution can be carried out at about room temperature. The dissolution can be carried out at essentially room temperature.

In some aspects, the readily dissolvable steviol glycoside composition can dissolve completely. The readily dissolvable steviol glycoside composition can dissolve completely within 1, 2, 3, 4, 5, 6, 7, 8, 9, 10, 11, 12, 13, 14, 15, 20, 25, 30, 35, 40, 45, 50, 55 or 60 minutes. The readily dissolvable steviol glycoside composition can dissolve completely within 1, 2, or 3 hours.

In some aspects, this disclosure also relates to readily dissolvable steviol glycoside compositions with a high rate of solution. An example of a readily dissolvable steviol glycoside composition with a high rate of solution is a composition comprising an admixture of a steviol glycoside and a dissolution enhancer compound in an amount effective to increase dissolution of the steviol glycoside. As described above, the term a high rate of solution can refer to a readily dissolvable composition and/or a composition with a high rate of dissolution in certain solvents. A high rate of solution can also refer to a composition comprising instantaneous solubility in certain solvents. For example, a steviol glycoside composition with a high rate of solution can comprise a high rate of dissolution and/or instantaneous solubility in a primarily aqueous solution.

In some aspects, the compositions comprising steviol glycoside and dissolution enhancer compound can comprise any suitable additives including but not limited to buffering agent, acidulants, such as citric acid, antimicrobial agents, such as benzoic acid and sorbic acid (and salts thereof), natural colors, natural flavors, artificial flavors, artificial colors, and artificial sweeteners.

In some aspects, the compositions comprising steviol glycoside and dissolution enhancer compound can comprise less than 0.3% (wt) of malonate, malonic acid, oxalate, oxalic acid, lactate, lactic acid, succinate, succinic acid, malate, or malic acid; or less than 0.05% (wt) of pyruvate, pyruvic acid, fumarate, fumaric acid, tartrate, tartaric acid, sorbate, sorbic acid, acetate, or acetic acid; or less than about 0.05% (wt) of chlorophyll.

In some aspects, the admixture of a steviol glycoside and a dissolution enhancer compound can comprise any steviol glycoside described above. For example, the admixed steviol glycoside can include rebaudioside M, rebaudioside D, rebaudioside A, rebaudioside B, rebaudioside O, and/or rebaudioside N. The admixed steviol glycoside can be in a crystalline form. Likewise, the admixed dissolution enhancer compound can comprise any suitable dissolution enhancer compound described above. For example, the admixed dissolution enhancer compound can include caffeic acid, monocaffeoylquinic acids (e.g. chlorogenic acid, neochlorogenic acid, and cryptochlorogenic acid), and dicaffeoylquinic acids (e.g. 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid), and salts thereof. The admixed dissolution enhancer compound can be prepared from any suitable source, including yerba mate, stevia, globe artichoke, and/or green coffee bean.

In some aspects, the admixture of the steviol glycoside and a dissolution enhancer compound can comprise any suitable ratio effective to increase dissolution of the steviol glycoside, as described above. For example, the amount of admixed dissolution enhancer compound effective to increase dissolution of the steviol glycoside can be an amount such that the dissolution enhancer compound comprises a ratio by weight of steviol glycoside to dissolution enhancer of about 1:0.1, 1:0.3, 1:0.5, 1:1, 1:2, 1:3, 1:4, 1:5, 1:6, 1:7, 1:8, 1:9, or 1:10 by weight.

The admixture of the steviol glycoside and a dissolution enhancer compound can be prepared by any suitable means to result in a composition with a high rate of solution. For example, dry steviol glycoside and dry dissolution enhancer compound can be combined to form a dry admixture. Likewise, a solution of steviol glycoside and dissolution enhancer compound can prepared and then dried to prepare the admixture. The admixture can comprise any other suitable ingredients. For example, the admixture can comprise a buffering system (e.g., a citrate/phosphate buffer). In some aspects, the buffering system can provide a pH of 0, 1, 2, 3, 4, 5, or 6. The pH can be between 1 and 6. The pH can be between 1.5 and 4.

In some aspects, the admixture of the steviol glycoside and the dissolution enhancer compound comprises an increased rate of dissolution in a primarily aqueous solution when compared to a steviol glycoside composition without a dissolution enhancer compound. The dissolution can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the dissolution can be increased by 1, 2, 3, 4, 5, 6, 7, 8, 9, or 10 times or more when compared to a composition without a dissolution enhancer compound.

In some aspects, the admixture of the steviol glycoside and the dissolution enhancer compound comprises an increased rate of solution in a primarily aqueous solution when compared to a steviol glycoside composition without a dissolution enhancer compound. The rate of solution can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the rate of solution can be increased by 1, 2, 3, 4, 5, 6, 7, 8, 9, or 10 times or more when compared to a composition without a dissolution enhancer compound.

In some aspects, the admixture of the steviol glycoside and the dissolution enhancer compound comprises an instantaneous solubility in a primarily aqueous solution when compared to a steviol glycoside composition without a dissolution enhancer compound. The instantaneous solubility can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the instantaneous solubility can be increased by 1, 2, 3, 4, 5, 6, 7, 8, 9, or 10 times or more when compared to a composition without a dissolution enhancer compound.

In some aspects, the admixture of the steviol glycoside and the dissolution enhancer compound comprises less than 1% stevioside. The admixture of the steviol glycoside and the dissolution enhancer compound can comprise less than 1% rebaudioside B. The admixture of the steviol glycoside and the dissolution enhancer compound can comprise less than 1% steviolbioside. The admixture of the steviol glycoside and the dissolution enhancer compound can comprise less than 1% 13-SMG. In other aspects, the admixture of the steviol glycoside and the dissolution enhancer compound comprises less than one or more of 1% stevioside, 1% rebaudioside B, 1% steviolbioside, and 1% 13-SMG.

In some aspects, the admixture of the steviol glycoside and the dissolution enhancer compound comprises an increased rate of solution in a primarily aqueous solution when compared to a steviol glycoside composition without a dissolution enhancer compound when a final concentration of steviol glycoside is between 3000 ppm and 60000 ppm. The final concentration of steviol glycoside can be greater than 100 ppm, 200 ppm, 300 ppm, 400 ppm, 500 ppm, 600 ppm, 700 ppm, 800 ppm, 900 ppm, or 1000 ppm. The final concentration of steviol glycoside can be greater than 1100 ppm, 1200 ppm, 1300 ppm, 1400 ppm, 1500 ppm, 1600 ppm, 1700 ppm, 1800 ppm, or 1900 ppm. The final concentration of steviol glycoside can be greater than 2100 ppm, 2200 ppm, 2300 ppm, 2400 ppm, 2500 ppm, 2600 ppm, 2700 ppm, 2800 ppm, or 2900 ppm. The final concentration of steviol glycoside can be greater than 3100 ppm, 3200 ppm, 3300 ppm, 3400 ppm, 3500 ppm, 3600 ppm, 3700 ppm, 3800 ppm, or 3900 ppm. The final concentration of steviol glycoside can be greater than 3000 ppm, 4000 ppm, 5000 ppm, 6000 ppm, 7000 ppm, 8000 ppm, or 9000 ppm. The final concentration of steviol glycoside can be greater than 1%, 2%, 3%, 4%, 5%, 6%, 7%, 8%, 9%, 10%, or higher.

In some aspects, the admixture of the steviol glycoside and the dissolution enhancer compound comprises an increased rate of solution in a primarily aqueous solution when compared to a steviol glycoside composition without a dissolution enhancer compound when a final concentration of dissolution enhancer compound is between 3000 ppm and 60000 ppm. The final concentration of dissolution enhancer compound can be greater than 100 ppm, 200 ppm, 300 ppm, 400 ppm, 500 ppm, 600 ppm, 700 ppm, 800 ppm, 900 ppm, or 1000 ppm. The final concentration of dissolution enhancer compound can be greater than 1100 ppm, 1200 ppm, 1300 ppm, 1400 ppm, 1500 ppm, 1600 ppm, 1700 ppm, 1800 ppm, or 1900 ppm. The final concentration of dissolution enhancer compound can be greater than 2100 ppm, 2200 ppm, 2300 ppm, 2400 ppm, 2500 ppm, 2600 ppm, 2700 ppm, 2800 ppm, or 2900 ppm. The final concentration of dissolution enhancer compound can be greater than 3100 ppm, 3200 ppm, 3300 ppm, 3400 ppm, 3500 ppm, 3600 ppm, 3700 ppm, 3800 ppm, or 3900 ppm. The final concentration of dissolution enhancer compound can be greater than 3000 ppm, 4000 ppm, 5000 ppm, 6000 ppm, 7000 ppm, 8000 ppm, or 9000 ppm. The final concentration of dissolution enhancer compound can be greater than 1%, 2%, 3%, 4%, 5%, 6%, 7%, 8%, 9%, 10%, or higher.

In some aspects, the admixture of the steviol glycoside and the dissolution enhancer compound comprises an increased rate of solution in a primarily aqueous solution when compared to a steviol glycoside composition without a dissolution enhancer compound when the dissolution is carried out at a temperature less than 5° C., 10° C., 15° C., 20° C., 25° C., 30° C., 35° C., 40° C., 45° C., 50° C., 55° C., 60° C., 65° C., 70° C., 75° C., 80° C., or 85° C. The dissolution can be carried out at between 5° C. and 65° C. The dissolution can be carried out between 20° C. and 65° C. The dissolution can be carried out at between 10° C. and 40° C. The dissolution can be carried out at between 15° C. and 30° C. The dissolution can be carried out at between 20° C. and 25° C. The dissolution can be carried out at about room temperature. The dissolution can be carried out at essentially room temperature.

In some aspects, the admixture of the steviol glycoside and the dissolution enhancer compound can dissolve completely. The admixture of the steviol glycoside and the dissolution enhancer compound can dissolve completely within 1, 2, 3, 4, 5, 6, 7, 8, 9, 10, 11, 12, 13, 14, 15, 20, 25, 30, 35, 40, 45, 50, 55 or 60 minutes. The admixture of the steviol glycoside and the dissolution enhancer compound can dissolve completely within 1, 2, or 3 hours.

In some aspects, this disclosure also relates to methods of increasing dissolution of crystalline steviol glycoside. An example of a method for increasing dissolution of a crystalline steviol glycoside, comprises contacting a crystalline steviol glycoside and a dissolution enhancer compound in an amount effective to increase dissolution of the steviol glycoside with a primarily aqueous solution.

In some aspects, the crystalline steviol glycoside can comprise any steviol glycoside described above. For example, the steviol glycoside can include crystalline forms of one or more of rebaudioside M, rebaudioside D, rebaudioside A, rebaudioside B, rebaudioside O, rebaudioside N, and/or stevioside. The dissolution enhancer compound can comprise any suitable dissolution enhancer compound described above. For example, the dissolution enhancer compound can include caffeic acid, monocaffeoylquinic acids (e.g. chlorogenic acid, neochlorogenic acid, and cryptochlorogenic acid), and dicaffeoylquinic acids (e.g. 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid), and salts thereof. The dissolution enhancer compound can be prepared from any suitable source, including yerba mate, stevia, globe artichoke, and/or green coffee.

In some aspects, the crystalline steviol glycoside and dissolution enhancer compound can comprise any suitable ratio, as described above. For example, the amount of admixed dissolution enhancer compound effective to increase dissolution of the steviol glycoside can be an amount such that the dissolution enhancer comprises a ratio by weight of dissolution enhancer compound to steviol glycoside of about 1:0.1, 1:0.3, 1:0.5, 1:1, 1:2, 1:3, 1:4, 1:5, 1:6, 1:7, 1:8, 1:9, or 1:10 by weight. the ratio of dissolution enhancer compound to steviol glycoside can be from about 0.1:1 to 10:1. In some aspects, the ratio of dissolution enhancer compound to steviol glycoside can be in the range of about 0.1:1 to 5:1, about 0.5:1 to 4:1, or about 1:1 to 3:1. In other aspects, the ratio of dissolution enhancer compound to steviol glycoside is about 0.1:1, 0.5:1, 1:1, 2:1, 3:1, 4:1, 5:1, 6:1, 7:1, 8:1, 9:1, or 10:1.

In some aspects, the rate of dissolution is increased in a primarily aqueous solution when compared to a steviol glycoside composition without a dissolution enhancer compound. The dissolution rate can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the dissolution rate can be increased by 1, 2, 3, 4, 5, 6, 7, 8, 9, or 10 times or more when compared to a composition without a dissolution enhancer compound.

In some aspects, the increased rate of dissolution corresponds to an increased rate of solution in a primarily aqueous solution when compared to a steviol glycoside composition without a dissolution enhancer compound. The rate of solution can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the rate of solution can be increased by 1, 2, 3, 4, 5, 6, 7, 8, 9, or 10 times or more when compared to a composition without a dissolution enhancer compound. In some aspects, the increased rate of dissolution corresponds to an increase in instantaneous solubility in a primarily aqueous solution when compared to a steviol glycoside composition without a dissolution enhancer compound. The instantaneous solubility can be increased by at least 5%, 10%, 15%, 20%, 25%, 30%, 35%, 40%, 45%, 50%, 55%, 60%, 65%, 70%, 75%, 80%, 85%, 90%, 95%, or 100% or more when compared to a composition without a dissolution enhancer compound. In other aspects, the instantaneous solubility can be increased by 1, 2, 3, 4, 5, 6, 7, 8, 9, or 10 times or more when compared to a composition without a dissolution enhancer compound.

In some aspects, the primarily aqueous solution in which the crystalline steviol glycoside is dissolved comprises primarily water. The primarily aqueous solution can also comprise less than 1%, 5%, 10%, 15%, 20%, 30%, 40%, 45%, 50%, 55%, 60%, 65%, 70%, 80%, or 90%, or about 40% to 65%, or about 50% to 55%, or about 55% of C1-C4 alcohol (e.g., methanol, ethanol, propanol, butanol, etc.). The primarily aqueous solution can be substantially free of C1-C4 alcohols. In other aspects, the primarily aqueous solution is essentially free of C1-C4 alcohols.

In some aspects, the primarily aqueous solution in which the crystalline steviol glycoside is dissolved comprises less than 1% stevioside. The primarily aqueous solution can comprise less than 1% rebaudioside B. The primarily aqueous solution can comprise less than 1% steviolbioside. The primarily aqueous solution can comprise less than 1% 13-SMG. In other aspects, the primarily aqueous solution comprises less than one or more of 1% stevioside, 1% rebaudioside B, 1% steviolbioside, and 1% 13-SMG.

In some aspects, the primarily aqueous solution in which the crystalline steviol glycoside is dissolved has any suitable pH. The primarily aqueous solution can also comprise a pH of 0, 1, 2, 3, 4, 5, or 6. The primarily aqueous solution can comprise a pH of between 0 and 7. The primarily aqueous solution can comprise a pH of between 1 and 6. The primarily aqueous solution can comprise a pH of between 1.5 and 4.

In some aspects, the method for increasing dissolution of a crystalline steviol glycoside comprises an increased rate of solution in a primarily aqueous solution when compared to a steviol glycoside composition without a dissolution enhancer compound when a final concentration of steviol glycoside is between 3000 ppm and 60000 ppm. The final concentration of steviol glycoside can be greater than 100 ppm, 200 ppm, 300 ppm, 400 ppm, 500 ppm, 600 ppm, 700 ppm, 800 ppm, 900 ppm, or 1000 ppm. The final concentration of steviol glycoside can be greater than 1100 ppm, 1200 ppm, 1300 ppm, 1400 ppm, 1500 ppm, 1600 ppm, 1700 ppm, 1800 ppm, or 1900 ppm. The final concentration of steviol glycoside can be greater than 2100 ppm, 2200 ppm, 2300 ppm, 2400 ppm, 2500 ppm, 2600 ppm, 2700 ppm, 2800 ppm, or 2900 ppm. The final concentration of steviol glycoside can be greater than 3100 ppm, 3200 ppm, 3300 ppm, 3400 ppm, 3500 ppm, 3600 ppm, 3700 ppm, 3800 ppm, or 3900 ppm. The final concentration of steviol glycoside can be greater than 3000 ppm, 4000 ppm, 5000 ppm, 6000 ppm, 7000 ppm, 8000 ppm, or 9000 ppm. The final concentration of steviol glycoside can be greater than 1%, 2%, 3%, 4%, 5%, 6%, 7%, 8%, 9%, 10%, or higher.

In some aspects, the method for increasing dissolution of a crystalline steviol glycoside comprises an increased rate of solution in a primarily aqueous solution when compared to a steviol glycoside composition without a dissolution enhancer compound when a final concentration of dissolution enhancer compound is between 3000 ppm and 60000 ppm. The final concentration of dissolution enhancer compound can be greater than 100 ppm, 200 ppm, 300 ppm, 400 ppm, 500 ppm, 600 ppm, 700 ppm, 800 ppm, 900 ppm, or 1000 ppm. The final concentration of dissolution enhancer compound can be greater than 1100 ppm, 1200 ppm, 1300 ppm, 1400 ppm, 1500 ppm, 1600 ppm, 1700 ppm, 1800 ppm, or 1900 ppm. The final concentration of dissolution enhancer compound can be greater than 2100 ppm, 2200 ppm, 2300 ppm, 2400 ppm, 2500 ppm, 2600 ppm, 2700 ppm, 2800 ppm, or 2900 ppm. The final concentration of dissolution enhancer compound can be greater than 3100 ppm, 3200 ppm, 3300 ppm, 3400 ppm, 3500 ppm, 3600 ppm, 3700 ppm, 3800 ppm, or 3900 ppm. The final concentration of dissolution enhancer compound can be greater than 3000 ppm, 4000 ppm, 5000 ppm, 6000 ppm, 7000 ppm, 8000 ppm, or 9000 ppm. The final concentration of dissolution enhancer compound can be greater than 1%, 2%, 3%, 4%, 5%, 6%, 7%, 8%, 9%, 10%, or higher.

In some aspects, the method for increasing dissolution of a crystalline steviol glycoside is carried out at a temperature less than 5° C., 10° C., 15° C., 20° C., 25° C., 30° C., 35° C., 40° C., 45° C., 50° C., 55° C., 60° C., 65° C., 70° C., 75° C., 80° C., or 85° C. The method for increasing dissolution of a crystalline steviol glycoside can be carried out at between 5° C. and 65° C. The dissolution can be carried out between 20° C. and 65° C. The dissolution can be carried out at between 10° C. and 40° C. The method for increasing dissolution of a crystalline steviol glycoside can be carried out at between 15° C. and 30° C. The method for increasing dissolution of a crystalline steviol glycoside can be carried out at between 20° C. and 25° C. The method for increasing dissolution of a crystalline steviol glycoside can be carried out at about room temperature. The method for increasing dissolution of a crystalline steviol glycoside can be carried out at essentially room temperature.

In some aspects, the crystalline steviol glycoside composition can dissolve completely. The crystalline steviol glycoside composition can dissolve completely within 1, 2, 3, 4, 5, 6, 7, 8, 9, 10, 11, 12, 13, 14, 15, 20, 25, 30, 35, 40, 45, 50, 55 or 60 minutes. The crystalline steviol glycoside composition can dissolve completely within 1, 2, or 3 hours.

In some aspects, this disclosure also relates to methods of preparing concentrated steviol glycoside solutions from crystalline steviol glycoside. An example of a method of preparing a concentrated steviol glycoside solution comprises dissolving a crystalline steviol glycoside and a dissolution enhancer compound in water, wherein a final concentration of the concentrated steviol glycoside solution is greater than 0.15% (wt), 0.2% (wt), 0.25% (wt), 0.3% (wt), 0.4% (wt), 0.5% (wt), 1% (wt), 3% (wt), 5% (wt), 10% (wt), 20% (wt), or greater.

In some aspects, the crystalline steviol glycoside can comprise any steviol glycoside described above. For example, the steviol glycoside can include crystalline forms of one or more of rebaudioside M, rebaudioside D, rebaudioside A, rebaudioside B, rebaudioside O, rebaudioside N, and/or stevioside. The dissolution enhancer compound can comprise any suitable dissolution enhancer compound described above. For example, the dissolution enhancer compound can include caffeic acid, monocaffeoylquinic acids (e.g. chlorogenic acid, neochlorogenic acid, and cryptochlorogenic acid), and dicaffeoylquinic acids (e.g. 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid), and salts thereof. The dissolution enhancer compound can be prepared from any suitable source, including yerba mate, stevia, globe artichoke, and/or green coffee.

In some aspects, the crystalline steviol glycoside and a dissolution enhancer compound can comprise any suitable ratio, as described above. For example, the ratio of dissolution enhancer compound to steviol glycoside can be from about 0.1:1 to 10:1. In some aspects, the ratio of dissolution enhancer compound to steviol glycoside can be in the range of about 0.1:1 to 5:1, about 0.5:1 to 4:1, or about 1:1 to 3:1. In other aspects, the ratio of dissolution enhancer compound to steviol glycoside is about 0.1:1, 0.3:1, 0.5:1, 0.7:1, 1:1, 2:1, 3:1, 4:1, 5:1, 6:1, 7:1, 8:1, 9:1, or 10:1.

In some aspects, the primarily aqueous solution in which the crystalline steviol glycoside is dissolved comprises primarily water. The primarily aqueous solution can also comprise less than 1%, 5%, 10%, 15%, 20%, 30%, 40%, 45%, 50%, 55%, 60%, 65%, 70%, 80%, or 90%, or about 40% to 65%, or about 50% to 55%, or about 55% of C1-C4 alcohol (e.g., methanol, ethanol, propanol, butanol, etc.). The primarily aqueous solution can be substantially free of C1-C4 alcohols. In other aspects, the primarily aqueous solution is essentially free of C1-C4 alcohols.

In some aspects, the primarily aqueous solution in which the crystalline steviol glycoside is dissolved comprises less than 1% stevioside. The primarily aqueous solution can comprise less than 1% rebaudioside B. The primarily aqueous solution can comprise less than 1% steviolbioside. The primarily aqueous solution can comprise less than 1% 13-SMG. In other aspects, the primarily aqueous solution comprises less than one or more of 1% stevioside, 1% rebaudioside B, 1% steviolbioside, and 1% 13-SMG.

In some aspects, the primarily aqueous solution in which the crystalline steviol glycoside is dissolved has any suitable pH. The primarily aqueous solution can also comprise a pH of 0, 1, 2, 3, 4, 5, or 6. The primarily aqueous solution can comprise a pH of between 0 and 7. The primarily aqueous solution can comprise a pH of between 1 and 6. The primarily aqueous solution can comprise a pH of between 1.5 and 4.

In some aspects, the concentrated steviol glycoside solution comprises a final concentration of steviol glycoside is between 3000 ppm and 60000 ppm. The final concentration of steviol glycoside can be greater than 100 ppm, 200 ppm, 300 ppm, 400 ppm, 500 ppm, 600 ppm, 700 ppm, 800 ppm, 900 ppm, or 1000 ppm. The final concentration of steviol glycoside can be greater than 1100 ppm, 1200 ppm, 1300 ppm, 1400 ppm, 1500 ppm, 1600 ppm, 1700 ppm, 1800 ppm, or 1900 ppm. The final concentration of steviol glycoside can be greater than 2100 ppm, 2200 ppm, 2300 ppm, 2400 ppm, 2500 ppm, 2600 ppm, 2700 ppm, 2800 ppm, or 2900 ppm. The final concentration of steviol glycoside can be greater than 3100 ppm, 3200 ppm, 3300 ppm, 3400 ppm, 3500 ppm, 3600 ppm, 3700 ppm, 3800 ppm, or 3900 ppm. The final concentration of steviol glycoside can be greater than 3000 ppm, 4000 ppm, 5000 ppm, 6000 ppm, 7000 ppm, 8000 ppm, or 9000 ppm. The final concentration of steviol glycoside can be greater than 1%, 2%, 3%, 4%, 5%, 6%, 7%, 8%, 9%, 10%, or higher.

In some aspects, the concentrated steviol glycoside solution comprises a final concentration of dissolution enhancer compound is between 3000 ppm and 60000 ppm. The final concentration of dissolution enhancer compound can be greater than 100 ppm, 200 ppm, 300 ppm, 400 ppm, 500 ppm, 600 ppm, 700 ppm, 800 ppm, 900 ppm, or 1000 ppm. The final concentration of dissolution enhancer compound can be greater than 1100 ppm, 1200 ppm, 1300 ppm, 1400 ppm, 1500 ppm, 1600 ppm, 1700 ppm, 1800 ppm, or 1900 ppm. The final concentration of dissolution enhancer compound can be greater than 2100 ppm, 2200 ppm, 2300 ppm, 2400 ppm, 2500 ppm, 2600 ppm, 2700 ppm, 2800 ppm, or 2900 ppm. The final concentration of dissolution enhancer compound can be greater than 3100 ppm, 3200 ppm, 3300 ppm, 3400 ppm, 3500 ppm, 3600 ppm, 3700 ppm, 3800 ppm, or 3900 ppm. The final concentration of dissolution enhancer compound can be greater than 3000 ppm, 4000 ppm, 5000 ppm, 6000 ppm, 7000 ppm, 8000 ppm, or 9000 ppm. The final concentration of dissolution enhancer compound can be greater than 1%, 2%, 3%, 4%, 5%, 6%, 7%, 8%, 9%, 10%, or higher.

In some aspects, the concentrated steviol glycoside solution is prepared at a temperature less than 5° C., 10° C., 15° C., 20° C., 25° C., 30° C., 35° C., 40° C., 45° C., 50° C., 55° C., 60° C., 65° C., 70° C., 75° C., 80° C., or 85° C. The concentrated steviol glycoside solution is prepared at between 5° C. and 65° C. The dissolution can be carried out between 20° C. and 65° C. The concentrated steviol glycoside solution is prepared at between 10° C. and 40° C. The concentrated steviol glycoside solution is prepared at between 15° C. and 30° C. The concentrated steviol glycoside solution is prepared at between 20° C. and 25° C. The concentrated steviol glycoside solution can be prepared at about room temperature. The concentrated steviol glycoside solution can be prepared at essentially room temperature.

In some aspects, the crystalline steviol glycoside composition can dissolve completely to make the concentrated steviol glycoside solution. The crystalline steviol glycoside composition can dissolve completely within 1, 2, 3, 4, 5, 6, 7, 8, 9, 10, 11, 12, 13, 14, 15, 20, 25, 30, 35, 40, 45, 50, 55 or 60 minutes. The crystalline steviol glycoside composition can dissolve completely within 1, 2, or 3 hours.

In some aspects, this disclosure also relates to methods of preparing a beverage concentrate from crystalline steviol glycoside. An example of a method for preparing a beverage concentrate comprises contacting a crystalline steviol glycoside, a dissolution enhancer compound, and water.

In some aspects, the crystalline steviol glycoside can comprise any steviol glycoside described above. For example, the steviol glycoside can include crystalline forms of one or more of rebaudioside M, rebaudioside D, rebaudioside A, rebaudioside B, rebaudioside N, and/or stevioside. The dissolution enhancer compound can comprise any suitable dissolution enhancer compound described above. For example, the dissolution enhancer compound can include caffeic acid, monocaffeoylquinic acids (e.g. chlorogenic acid, neochlorogenic acid, and cryptochlorogenic acid), and dicaffeoylquinic acids (e.g. 1,3-dicaffeoylquinic acid, 1,4-dicaffeoylquinic acid, 1,5-dicaffeoylquinic acid, 3,4-dicaffeoylquinic, 3,5-dicaffeoylquinic acid, and 4,5-dicaffeoylquinic acid), and salts thereof. The dissolution enhancer compound can be prepared from any suitable source, including stevia and/or yerba mate.

In some aspects, the crystalline steviol glycoside and a dissolution enhancer compound can comprise any suitable ratio, as described above. For example, the ratio of dissolution enhancer compound to steviol glycoside can be from about 0.1:1 to 10:1. In some aspects, the ratio of dissolution enhancer compound to steviol glycoside can be in the range of about 0.1:1 to 5:1, about 0.5:1 to 4:1, or about 1:1 to 3:1. In other aspects, the ratio of dissolution enhancer compound to steviol glycoside is about 0.1:1, 0.3:1, 0.5:1, 0.7:1, 1:1, 2:1, 3:1, 4:1, 5:1, 6:1, 7:1, 8:1, 9:1, or 10:1.

In some aspects, the beverage concentrate comprises less than 1%, 5%, 10%, 15%, 20%, 30%, 40%, 45%, 50%, 55%, 60%, 65%, 70%, 80%, or 90%, or about 40% to 65%, or about 50% to 55%, or about 55% of C1-C4 alcohol (e.g., methanol, ethanol, propanol, butanol, etc.). The beverage concentrate can be substantially free of C1-C4 alcohols. In other aspects, the beverage concentrate is essentially free of C1-C4 alcohols. In other aspects, the beverage concentrate in which the crystalline steviol glycoside is dissolved comprises less than 1% stevioside. The water can comprise less than 1% rebaudioside B. The beverage concentrate can comprise less than 1% rebaudioside F. The beverage concentrate can comprise less than 1% rebaudioside C. In other aspects, the beverage concentrate comprises less than one or more of 1% stevioside, 1% rebaudioside B, 1% rebaudioside F, 1% steviolbioside, 1% 13-SMG, and 1% rebaudioside C.

In some aspects, the beverage concentrate comprises a final concentration of steviol glycoside is between 3000 ppm and 60000 ppm. The final concentration of steviol glycoside can be greater than 100 ppm, 200 ppm, 300 ppm, 400 ppm, 500 ppm, 600 ppm, 700 ppm, 800 ppm, 900 ppm, or 1000 ppm. The final concentration of steviol glycoside can be greater than 1100 ppm, 1200 ppm, 1300 ppm, 1400 ppm, 1500 ppm, 1600 ppm, 1700 ppm, 1800 ppm, or 1900 ppm. The final concentration of steviol glycoside can be greater than 2100 ppm, 2200 ppm, 2300 ppm, 2400 ppm, 2500 ppm, 2600 ppm, 2700 ppm, 2800 ppm, or 2900 ppm. The final concentration of steviol glycoside can be greater than 3100 ppm, 3200 ppm, 3300 ppm, 3400 ppm, 3500 ppm, 3600 ppm, 3700 ppm, 3800 ppm, or 3900 ppm. The final concentration of steviol glycoside can be greater than 3000 ppm, 4000 ppm, 5000 ppm, 6000 ppm, 7000 ppm, 8000 ppm, or 9000 ppm. The final concentration of steviol glycoside can be greater than 1%, 2%, 3%, 4%, 5%, 6%, 7%, 8%, 9%, 10%, or higher.

In some aspects, the beverage concentrate comprises a final concentration of dissolution enhancer compound is between 3000 ppm and 60000 ppm. The final concentration of dissolution enhancer compound can be greater than 100 ppm, 200 ppm, 300 ppm, 400 ppm, 500 ppm, 600 ppm, 700 ppm, 800 ppm, 900 ppm, or 1000 ppm. The final concentration of dissolution enhancer compound can be greater than 1100 ppm, 1200 ppm, 1300 ppm, 1400 ppm, 1500 ppm, 1600 ppm, 1700 ppm, 1800 ppm, or 1900 ppm. The final concentration of dissolution enhancer compound can be greater than 2100 ppm, 2200 ppm, 2300 ppm, 2400 ppm, 2500 ppm, 2600 ppm, 2700 ppm, 2800 ppm, or 2900 ppm. The final concentration of dissolution enhancer compound can be greater than 3100 ppm, 3200 ppm, 3300 ppm, 3400 ppm, 3500 ppm, 3600 ppm, 3700 ppm, 3800 ppm, or 3900 ppm. The final concentration of dissolution enhancer compound can be greater than 3000 ppm, 4000 ppm, 5000 ppm, 6000 ppm, 7000 ppm, 8000 ppm, or 9000 ppm. The final concentration of dissolution enhancer compound can be greater than 1%, 2%, 3%, 4%, 5%, 6%, 7%, 8%, 9%, 10%, or higher.

In some aspects, the beverage concentrate is prepared at a temperature less than 5° C., 10° C., 15° C., 20° C., 25° C., 30° C., 35° C., 40° C., 45° C., 50° C., 55° C., 60° C., 65° C., 70° C., 75° C., 80° C., or 85° C. The beverage concentrate is prepared at between 5° C. and 65° C. The dissolution can be carried out between 20° C. and 65° C. The beverage concentrate is prepared at between 10° C. and 40° C. The beverage concentrate is prepared at between 15° C. and 30° C. The beverage concentrate is prepared at between 20° C. and 25° C. The beverage concentrate can be prepared at about room temperature. The beverage concentrate can be prepared at essentially room temperature.

In some aspects, the crystalline steviol glycoside composition can dissolve completely to make the beverage concentrate. The crystalline steviol glycoside composition can dissolve completely within 1, 2, 3, 4, 5, 6, 7, 8, 9, 10, 11, 12, 13, 14, 15, 20, 25, 30, 35, 40, 45, 50, 55 or 60 minutes. The crystalline steviol glycoside can dissolve completely within 1, 2, or 3 hours.

The beverage concentrate can further comprise a buffer (e.g., a citrate/phosphate buffer). In some aspects, the buffer can provide a pH of 0, 1, 2, 3, 4, 5, or 6. The pH can be between 1 and 6. The pH can be between 1.5 and 4.

In some aspects, the beverage concentrate can further comprise any suitable flavor and/or color. In some aspects, the beverage concentrate can further comprise any suitable ingredient including buffering agent, acidulants, such as citric acid, antimicrobial agents, such as benzoic acid and sorbic acid (and salts thereof), natural colors, natural flavors, artificial flavors, artificial colors, and artificial sweeteners.

### EXAMPLES

The following examples are provided to illustrate the disclosure, but are not intended to limit the scope thereof. All parts and percentages are by weight unless otherwise indicated.

### Example 1

A series of dissolution assays were carried out on the dissolution of a steviol glycoside blend with and without dissolution enhancer compound. The dissolution assays were performed in an acidic citrate/phosphate buffer system similar to that of a carbonated soda beverage concentrate (or throw syrup). One steviol glycoside blend comprised primarily rebaudioside D and rebaudioside M (RM80, >80% (wt) rebaudioside M). Another steviol glycoside blend comprised a 95% rebaudioside A blend (RA95). The dissolution enhancer compounds were prepared from stevia leaf and also from yerba mate.

An aqueous solution of citrate/phosphate buffer (about 4 g/L) was prepared and adjusted to pH 2.5. Separate capped glass vials were prepared, 10 mL of the aqueous buffered solution was aliquoted into each vial, and a magnetic stir bar added to each vial. Dry powdered samples of steviol glycoside blends were added to some vials under magnetic stirring. Dry powdered samples of steviol glycoside blends along with dry powdered samples of dissolution enhancer compounds were added to other vials under magnetic stirring. Each vial was monitored over time for dissolution under magnetic stirring and the results recorded by video or still image.

A control vial was prepared with 10 mL of the aqueous buffered solution as described above. A dry powdered sample of the RM80 corresponding to a final steviol glycoside concentration of 3000 ppm was added to the aqueous buffered solution under magnetic stirring at room temperature. Magnetic stirring was continued and dissolution monitored over time. The initially clear aqueous buffered solution became cloudy upon addition of the RM80. At 1 minute after addition of the RM80 the aqueous buffered solution remained cloudy. At 10 minutes after addition of the RM80 the aqueous buffered solution remained cloudy. At 45 minutes after addition of the RM80 the aqueous buffered solution remained cloudy. At 1440 minutes after addition of the RM80 the aqueous buffered solution remained cloudy. Table 1 tabulates the observations of the control vial at 0 minutes, 1 minute, 10 minutes, 45 minutes, 60 minutes, 120 minutes, 300 minutes, and 1440 minutes, respectively.

TABLE 1
Observations of control vial over time

| Time (min) | Observation |
|---|---|
| 0 | Cloudy |
| 1 | Cloudy |
| 10 | Cloudy |
| 45 | Cloudy |
| 60 | Cloudy |
| 120 | Cloudy |
| 300 | Cloudy |
| 1440 | Cloudy |

Table 1 shows that the RM80 added to the aqueous buffered solution remained cloudy over the course of the monitoring. This control vial experiment showed that the RM80 at a final steviol glycoside concentration of 3000 ppm did not dissolve completely in the aqueous buffered solution.

### Example 2

A vial was prepared with 10 mL of the aqueous buffered solution as described above. A dry powdered sample of the RM80 corresponding to a final steviol glycoside concentration of 3000 ppm was added to the aqueous buffered solution under magnetic stirring at room temperature. A dry powdered sample of the dissolution enhancer compound prepared from stevia corresponding to a final concentration of 3000 ppm was also added to the aqueous buffered solution under magnetic stirring at room temperature. Magnetic stirring was continued and dissolution monitored over time. The initially clear aqueous buffered solution became cloudy upon addition of the RM80. At 1 minute after addition of the RM80 the aqueous buffered solution became slightly less cloudy. At 3 minutes after addition of the RM80 the aqueous buffered solution became less cloudy. At 11 minutes after addition of the RM80 the aqueous buffered solution was clear with a few steviol glycoside crystals remaining. At 15 minutes after addition of the RM80 the aqueous buffered solution was completely clear with no steviol glycoside crystals remaining. Table 2 tabulates observations of the vial at 0 minutes, 1 minute, 3 minutes, 11 minutes, 15 minutes, 60 minutes, and 1440 minutes, respectively.

TABLE 2
Observations of test vial over time

| Time (min) | Observation |
|---|---|
| 0 | Cloudy |
| 1 | Slightly less cloudy |
| 3 | Less cloudy |
| 11 | Clear with few remaining |
| | steviol glycoside crystals |
| 15 | Completely clear |
| 60 | Completely clear |
| 1440 | Completely clear |

Table 2 shows that the RM80 added to the aqueous buffered solution with the dissolution enhancer compounds dissolved completely over the course of the monitoring. This experiment showed that the combination of the RM80 at a final steviol glycoside concentration of 3000 ppm and the dissolution enhancer compound from stevia at a final concentration of 3000 was able to readily dissolve in the aqueous buffered solution

### Example 3

A vial was prepared with 10 mL of the aqueous buffered solution as described above. A dry powdered sample of the RM80 corresponding to a final steviol glycoside concentration of 5000 ppm was added to the aqueous buffered solution under magnetic stirring at room temperature. A dry powdered sample of the dissolution enhancer compound prepared from stevia corresponding to a final concentration of 5000 ppm was also added to the aqueous buffered solution under magnetic stirring at room temperature. Magnetic stirring was continued and dissolution monitored over time. The initially clear aqueous buffered solution became cloudy upon addition of the RM80. At 10 minutes after addition of the RM80 the aqueous buffered solution became less cloudy. At 2 hours after addition of the RM80 the aqueous buffered solution was completely clear with no steviol glycoside crystals remaining. Table 3 lists observations of the test vial at 0 minutes, 10 minutes, 2 hours, and 24 hours, respectively.

TABLE 3

| Time (min) | Observation |
|---|---|
| 0 | Cloudy |
| 10 | Less cloudy |
| 120 | Completely clear |
| 1440 | Completely clear |

Table 3 shows that the RM80 added to the aqueous buffered solution with the dissolution enhancer compounds dissolved completely over the course of the monitoring. This experiment showed that the combination of the RM80 at a final steviol glycoside concentration of 5000 ppm and the dissolution enhancer compound from stevia at a final concentration of 5000 was able to dissolve completely in the aqueous buffered solution.

### Example 4

A vial was prepared with 10 mL of the aqueous buffered solution as described above. A dry powdered sample of the RM80 corresponding to a final steviol glycoside concentration of 3000 ppm was added to the aqueous buffered solution under magnetic stirring at room temperature. A dry powdered sample of the dissolution enhancer compound prepared from yerba mate corresponding to a final concentration of 3000 ppm was also added to the aqueous buffered solution under magnetic stirring at room temperature. Magnetic stirring was continued and dissolution monitored over time. The initially clear aqueous buffered solution became cloudy upon addition of the RM80. At 5 minutes after addition of the RM80 the aqueous buffered solution became less cloudy. At 15 minutes after addition of the RM80 the aqueous buffered solution was less cloudy with some steviol glycoside crystals remaining. At 2 hours after addition of the RM80 the aqueous buffered solution was completely clear with no steviol glycoside crystals remaining. Table 4 shows observations of the test vial at 0 minutes, 5 minutes, 15 minutes, 2 hours, and 24 hours respectively.

TABLE 4

| Time (min) | Observation |
|---|---|
| 0 | Cloudy |
| 5 | Less cloudy |
| 15 | Less cloudy with some steviol |
| | glycoside crystals remaining |
| 120 | Completely clear |
| 1440 | Completely clear |

The RM80 added to the aqueous buffered solution with the dissolution enhancer compounds dissolved completely over the course of the monitoring. This experiment showed that the combination of the RM80 at a final steviol glycoside concentration of 3000 ppm and the dissolution enhancer compound from yerba mate at a final concentration of 3000 ppm was able to dissolve completely in the aqueous buffered solution.

### Example 5

A vial was prepared with 10 mL of the aqueous buffered solution at pH 4 (citrate buffer, about 4 g/L). A dry powdered sample of the dissolution enhancer compound prepared from stevia and corresponding to a final concentration of 60000 ppm (6%) was added to the aqueous buffered solution at room temperature and the vial was capped and vortexed until the solubility enhancer dissolved. A dry powdered sample of 95% Rebaudioside A blend (RA95) corresponding to a final concentration of 60000 ppm (6%) was then added to the vial and the vial was capped and vortexed for 4 seconds. After vortexing, the aqueous buffered solution was observed for dissolution of the RA95. The RA95 had dissolved into solution and the aqueous buffered solution was clear. The RA95 blend added to the aqueous buffered solution with the dissolution enhancer compounds dissolved completely after vortexing. This experiment showed that the RA95 blend at a final steviol glycoside concentration of 60000 ppm (6%) was able to dissolve completely in the aqueous buffered solution containing the dissolution enhancer compound from stevia at a final concentration of 60000 ppm (6%).

TABLE 5

| Time (min) | Observation |
|---|---|
| 0 | Cloudy |
| 0.10 | Less cloudy |
| 0.15 | Less cloudy with some steviol |
| | glycoside crystals remaining |
| 0.20 | Completely clear |

### Example 6

A 1:1 (wt) solution of the RM80 and the dissolution enhancer compound was prepared and co-dried to produce a dry 1:1 (wt) mixture. RM80 was added to a control vial. The 1:1 (wt) mixture of the RM80 and the dissolution enhancer compound was added to a test vial. Room temperature water was added to each vial. The room temperature water was added to the control vial in an amount to yield a 5% (wt) solution of the RM80. The room temperature water was added to the test vial in an amount to yield a solution of 5% (wt) RM80 and 5% (wt) SG dissolution enhancer. The vials were capped immediately after the water was added and each vial shaken by hand for about 10 seconds and observed immediately after shaking. The control vial showed that the RM80 did not dissolve. The test vial showed that the 1:1 (wt) mixture of the RM80 and the dissolution enhancer compound dissolved. The solutions were monitored over time. The solution of the test vial remained dissolved after 2 weeks of time. This experiment showed that the RM80 with dissolution enhancer compound was able to dissolve. The experiment also showed a high rate of instantaneous solubility of the RM80 with dissolution enhancer compound.

### Example 7

Several vials were prepared to examine the effect of temperature on the dissolution of Reb M in water. For each experiment, a vial was prepared with an appropriate amount of steviol glycoside powder (RM80 or RM90). Dissolution enhancer was added to the vials as described in Table 6. Water was added to each vial to achieve the final concentrations described in Table 6. The vials were mixed by hand and placed in warm water baths at increasing temperatures. The minimum temperature required to dissolve the steviol glycosides is recorded in Table 6 as well as the time required at that temperature to reach complete dissolution.

TABLE 6

| | SG | Dissolution | | | Time for |
|---|---|---|---|---|---|
| | concen- | enhancer | | Temperature | complete |
| Reb | tration | concentration | SE:SG | required | dissolution |
| M Lot | (ppm) | (ppm) | Ratio | (° C.) | (minutes) |
| Lot #2 | 270 | 0 | — | 65 | 2 |
| Lot #2 | 270 | 270 | 1:1 | 20 | 2 |
| Lot #2 | 450 | 0 | — | 65 | 2 |
| Lot #2 | 450 | 300 | 2:3 | 20 | 2 |
| Lot #1 | 500 | 0 | — | 65 | 6 |
| Lot #1 | 500 | 250 | 1:2 | 20 | 4.5 |
| Lot #1 | 500 | 375 | 3:4 | 20 | 4.5 |
| Lot #2 | 600 | 0 | — | 65 | 2 |
| Lot #2 | 600 | 400 | 2:3 | 20 | 2 |
| Lot #2 | 700 | 0 | — | 65 | 2 |
| Lot #2 | 700 | 475 | 2:3 | 20 | 2 |
| Lot #1 | 2990 | 0 | — | 65 | 12 |
| Lot #1 | 2990 | 1500 | 1:2 | 40 | 10 |
| Lot #1 | 2990 | 2240 | 3:4 | 20 | 4 |

1.-44. (canceled)

45. A method for preparing a steviol glycoside solution, the method comprising:
adding to an aqueous solution rebaudioside M; one or more dicaffeoylquinic acids or salts thereof; and one or more monocaffeoylquinic acids or salts thereof to form the steviol glycoside solution,
wherein rebaudioside M is at least 70% by weight of total steviol glycosides in the steviol glycoside solution; and
wherein when added to an aqueous solution to from a 5 wt % steviol glycoside solution, the rebaudioside M is completely dissolved in less than 10 seconds.

46. The method of claim 45, wherein the ratio of total steviol glycosides to total of monocaffeoylquinic acids or salts and dicaffeoylquinic acids and salts is between 1:0.3 to 1:3 in the steviol glycoside solution.

47. The method of claim 45, wherein the ratio of total steviol glycosides to total of monocaffeoylquinic acids or salts and dicaffeoylquinic acids and salts is between 1:1 to 1:3 in the steviol glycoside solution.

48. The method of claim 45, wherein the one or more dicaffeoylquinic acids or salts thereof comprise one or more of 1,3-dicaffeoylquinic acid, 1,4-dicaffeoylquinic acid, 1,5-dicaffeoylquinic acid, 3,4-dicaffeoylquinic acid, 3,5-dicaffeoylquinic acid, 4,5-dicaffeoylquinic acid, and salts thereof.

49. The method of claim 45, wherein the one or more monocaffeoylquinic acids or salts thereof comprise one or more of 3-O-caffeoylquinic acid, 4-O-caffeoylquinic acid, 5-O-caffeoylquinic acid, and salts thereof.

50. The method of claim 45, wherein the steviol glycoside is crystalline.

51. The method of claim 45, wherein the steviol glycoside comprises rebaudioside D.

52. The method of claim 45, wherein the steviol glycoside comprises rebaudioside A.

53. The method of claim 45, wherein the steviol glycoside, the one or more dicaffeoylquinic acid or salt thereof and the one or more monocaffeoylquinic acid or salt thereof are co-dried prior to addition to the aqueous solution.

54. The method of claim 45, wherein the one or more dicaffeoylquinic acids or salts thereof and the one or more monocaffeoylquinic acids or salts thereof are prepared from a botanical source.

55. The method of claim 54, wherein the botanical source is yerba mate.

56. The method of claim 54, wherein the botanical source is stevia.

57. The method of claim 45, wherein the total of monocaffeoylquinic acids or salts and dicaffeoylquinic acids and salts comprises at least 15% dicaffeoylquinic acids and salts thereof.

58. The method of claim 45, wherein at least one of 3-O-feruloylquinic acid, 4-O-feruloylquinic acid, 5-O-feruloylquinic acid, 3,4-diferuloylquinic acid, 3,5-diferuloylquinic acid, or 4,5-diferuloylquinic acid is also added to the aqueous solution.

59. The method of claim 45, wherein the aqueous solution comprises less than 1% C1-C4 alcohols.

60. The method of claim 45, wherein the aqueous solution is substantially free of C1-C4 alcohols.

61. The method of claim 45, wherein the steviol glycoside dicaffeoylquinic acids or salts thereof, and monocaffeoylquinic acids or salts thereof are added to the aqueous solution at a temperature less than 30° C., the total steviol glycoside concentration is at least 5 wt % in the steviol glycoside solution, and the rebaudioside M completely dissolves in less than 10 seconds.

62. The method of claim 45, wherein the final concentration of steviol glycoside in the steviol glycoside solution is between 3000 ppm and 60000 ppm.

63. The method of claim 45, wherein total steviol glycoside in the steviol glycoside solution comprises less than 1 wt % of rebaudioside A.

64. The method of claim 45, wherein total steviol glycoside in the steviol glycoside solution comprises less than 1 wt % rebaudioside A, at least 80 wt % rebaudioside M, and between 10 wt % and 20 wt % rebaudioside D.
